(19) United States
(12) Patent Application Publication
(10) Pub. No.: US 2026/0079143 A1
(43) Pub. Date: Mar. 19, 2026

(54) SYSTEM AND METHOD OF PLUME MODELING

(71) Applicant: Industrial Scientific Corporation, Pittsburgh, PA (US)

(72) Inventors: Timothy B. Thompson, Chicago, IL (US); Gregory J. Persson, Pittsburgh, PA (US); Venkatesu Punugupati, Cary, NC (US); Andrew Fisher, Sarasota, FL (US); Adam Daniels, Raleigh, NC (US); Pronitha Shankarananda, San Jose, CA (US); Dante A. Moore, New Brighton, PA (US); Sachin Shah, Troy, MI (US)

(21) Appl. No.: 19/401,947

(22) Filed: Nov. 26, 2025

Related U.S. Application Data
(63) Continuation of application No. PCT/US2025/041137, filed on Aug. 7, 2025.
(60) Provisional application No. 63/680,434, filed on Aug. 7, 2024.
(60) Provisional application No. 63/681,510, filed on Aug. 9, 2024.
(60) Provisional application No. 63/712,661, filed on Oct. 28, 2024.

(51) Int. Cl. G01N 33/00

(52) U.S. Cl. CPC G01N 33/0073

(57) ABSTRACT

A system includes a controller including a gas monitor data circuit structured to interpret gas monitoring data from gas monitor end points of a cooperative monitoring group associated with a facility, a plume characterization circuit structured to determine a plume configuration comprising a gas constituent distribution in response to the gas monitoring data, and a plume reporting circuit structured to provide a plume communication in response to the plume configuration.

### CROSS-REFERENCE TO RELATED APPLICATIONS

This application claims priority to and is a continuation of International Patent Application Serial No. PCT/US2025/041137, filed Aug. 7, 2025 (Attorney Docket No. ISCI-0059-WO).

International Patent Application Serial No. PCT/US2025/041137 claims the benefit of and priority to U.S. Application Ser. No. 63/680,434 , filed on Aug. 7, 2024 (Attorney Docket No. ISCI-0054-P01), U.S. Application Ser. No. 63/681,510 , filed on Aug. 9, 2024 (Attorney Docket No. ISCI-0055-P01), and U.S. Application Ser. No. 63/712,661 , filed on Oct. 28, 2024 (Attorney Docket No. ISCI-0056-P01). Each of the foregoing applications are incorporated herein by reference in their entireties for all purposes.

The following PCT International applications are each incorporated by reference herein in their entireties for all purposes: International Application Serial No. PCT/US2025/040942, filed Aug. 6, 2025 (Attorney Docket No.: ISCI-0057-WO); and International Application Serial No. PCT/US2025/040966, filed Aug. 6, 2025 (Attorney Docket No. ISCI-0058-WO).

### BACKGROUND

Gas monitoring at various types of industrial facilities is crucial to maintaining a safe environment, protecting workers, and ensuring proper operation of facilities. Previously known gas monitoring systems suffer from a number of drawbacks. For example, increasing electronic demands on monitoring devices compete with the desire for long battery life, and reduction of time spent on power management of gas monitors. In another example, devices need to be calibrated and maintained, and the workflows to perform these operations are burdensome to the ongoing operation of the facility and for individual operators using a gas monitor. Previously known systems do not provide for convenient and ready monitoring environments that can support arbitrary monitoring, for example in locations such as enclosed spaces, that have high and convenient availability, and that are integrated into the general alarm system for the gas monitoring system. Further, previously known systems are challenged by the flexibility required to support different industrial facilities with different constraints and implementation challenges, where significant challenges are raised in designing systems to meet the needs of some facilities without forcing excessive cost into the solution for features that are not needed.

### SUMMARY

The disclosure herein provides numerous benefits with respect to addressing the challenges of managing gas monitoring of industrial facilities. Embodiments herein support maintaining gas monitors at a facility, including operations to distribute gas monitors to the facility to maintain sufficient gas monitors at the facility for proper operations, to replace gas monitors that have faults, failed sensing elements, and/or that are otherwise unable to support gas monitoring operations at the facility, to expand the number of gas monitors, and/or to support rapid and/or temporary deployments of gas monitors for various purposes such as providing a rapid fence-line, monitoring for temporary processes and/or events at the facility. Such embodiments, without limitation, reduce gas monitor downtime, seamlessly maintain sufficient gas monitors for operations, and improve compliance and safety at the facility. Embodiments herein support improved worker exposure determination, including estimating exposure events that are not detected by a gas monitor utilized by workers, providing a convenient overview of exposure to gases, and supporting flexibility in gas monitor utilization by the worker to ensure that exposure events are detected on behalf of the worker. Embodiments herein support ease of gas monitoring operations and improve coverage, supporting operations to maintain monitoring capability at distance and/or difficult locations, allowing for rapid installation, and providing for rapid changes in the monitoring network configuration, which ensure that gas monitoring coverage can be provided during facility changes, and reduce facility downtime. Embodiments herein provide for convenient modeling and analysis of facility gas exposure, confident determination that exposure risks are managed and/or detected where present, and assist in planning facility layouts, evacuation planning, and evacuation execution to reduce the overall risk profile of the facility. Embodiments herein reduce the costs to manage and respond to false gas alerts, and risks from undetected gas alerts.

These and other systems, methods, objects, features, and advantages of the present disclosure will be apparent to those skilled in the art from the following detailed description of the preferred embodiment and the drawings.

### BRIEF DESCRIPTION OF THE FIGURES

FIG. 1 depicts an example system including a facility with gas monitoring.

FIG. 2 depicts an example controller to support gas monitor distribution support.

FIG. 3 depicts example gas monitor status values.

FIG. 4 depicts an example controller to support exposure tracking.

FIG. 5 depicts an example exposure description.

FIG. 6 depicts an example controller to support monitor network bridging and extension.

FIG. 7 depicts example monitoring communications.

FIG. 8 depicts an example controller to support alert communications.

FIG. 9 depicts example alert communications.

FIG. 10 depicts an example controller to support plume modeling and visualization.

FIG. 11 depicts example plume configurations.

FIG. 12 depicts example external data utilized in plume modeling and related operations.

FIG. 13 depicts an example plume display.

FIG. 14 depicts another example plume display.

FIG. 15 depicts another example plume display.

### DETAILED DESCRIPTION

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

Before the present disclosure is described in further detail, it is to be understood that the disclosure is not limited to the particular embodiments described. It is also understood that the terminology used herein is for the purpose of describing particular embodiments only, and is not intended to be limiting. The scope of the present disclosure will be limited only by the claims. As used herein, the singular forms “a”, “an”, and “the” include plural embodiments unless the context clearly dictates otherwise.

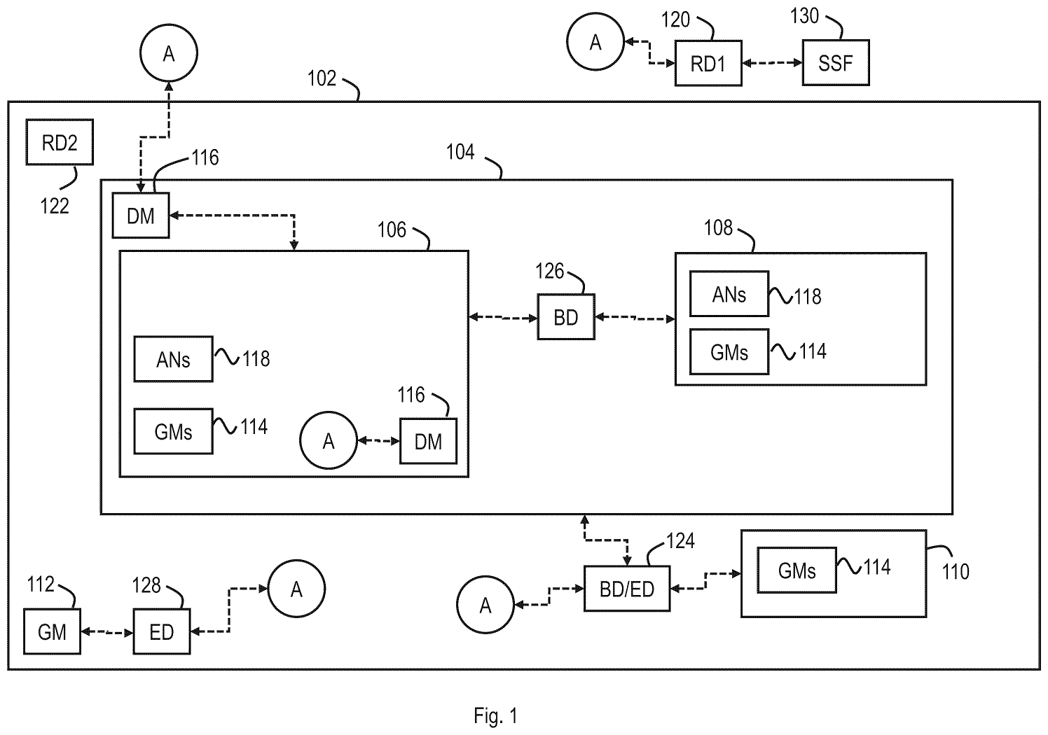

Referencing FIG. 1 an example system is depicted, including a facility 102 having gas monitoring that may utilize aspects of the present disclosure as set forth throughout. The example facility may be any type of facility where gas monitoring is relevant, and a typical gas monitoring arrangement includes individual monitors worn by, and/or associated with, workers at the facility. Additionally or alternatively, the gas monitors may be positioned at locations in the facility to monitor gases associated with an area. The example facility 102 includes a cooperative monitoring group 104, including a number of monitors that collectively monitor gases at the facility and/or at locations where workers having gas monitors are located. The cooperative monitoring group 104 includes a number of monitors that may be evaluated together, individually, and/or in selected groups (e.g., by location, by a team of people related in some manner such as in a related workflow, or the like), to enhance the capabilities of the gas monitoring system as set forth herein. The example cooperative monitoring group 104 includes a number of end points operating, at least in part, as a low power wireless mesh network, where individual monitors and/or other end points operate as nodes on the mesh network and support communications between devices, and/or to remote devices such as a facility remote device 122 (e.g., utilized by a facility owner, operator, manager, and/or on-site safety personal) and/or an external remote device 120 (e.g., a cloud server, personnel accessing the system remotely for monitoring operations, such as through a WAN connection, the internet, accessing a cloud server, web portal, mobile device, or the like). The example cooperative monitoring group 104 includes a first selected group of monitors 106 and a second selected group of monitors 108 that are communicatively coupled using a bridging device 126 such as an independently powered gas monitor capable of operating for extended periods without external power. The bridging device 126 in the example allows for connection of the mesh network portions 106, 108 without additional infrastructure, and while limiting the number of expensive and/or high maintenance gas monitors that must be present at the facility 102 to support monitoring operations. Another example cooperative monitoring group 104 includes an additional monitor 112 positioned at a location that is not communicatively coupled to the network portions 106, 108, for example due to distance and/or environmental challenges (e.g., underground, with interfering metal components, etc.), and a broadcast capable monitor 128 (e.g., having communication components capable of reaching remote devices 120, 122, such as a cellular transceiver and/or satellite transceiver) that communicatively couples the additional monitor 112 (or more than one additional monitor) to the rest of the system. The broadcast capable monitor 128 allows for extension of the network to challenging and/or distant locations without additional supporting infrastructure, and limiting the number of expensive and/or high maintenance gas monitors that must be present at the facility 102. In the example of FIG. 1, the additional monitor 112 may be considered as a part of the cooperative monitoring group 104, at least during certain time periods and/or operating conditions. Another example in FIG. 1 includes a high capability monitoring device 124, for example including extended independent power and/or broadcast capability, that supports communications for another selected group of monitors 110, which selectively bridges the group 110 to the other groups 106, 108, and/or communicatively couples the group 110 to the remote device 120 and/or to the groups 106, 108 through the remote device 120. In the example of FIG. 1, the group 110 may be considered as a part of the cooperative monitoring group 104, at least during certain time periods and/or operating conditions. The examples of FIG. 1 are illustrative of aspects of the system to depict various capabilities of embodiments of the present disclosure, and are non-limiting.

In the example of FIG. 1, a mesh network may include anchors 118, for example capable of acting as nodes on the mesh network, which can be operated on battery power only for extended periods, and provide fixed and reliable communication points that may be useful, for example, when the monitors 114 at the facility are moved around and might otherwise create gaps in the available mesh nodes. The example system may further include gas station components (not shown), for example stations allowing for charging, diagnostic operations, calibration operations, firmware updates, or the like, and which may optionally operate as nodes on the mesh network. In the example of FIG. 1, external communications for the cooperative monitoring group 104 may be limited to data manager components 116 and/or to broadcast capable devices 128, 124, limiting the infrastructure requirements (e.g., providing power and/or network connectivity at various locations in the facility 102) and/or simplifying communications with remote devices 120, 122, as well as allowing high count devices such as monitors 114 and anchors 118 to avoid infrastructure support, and to be provided without expensive or heavy components such as cellular or satellite capability and high capacity battery packs. The example system further includes a service and supply facility 130, which may not be limited to a single facility, where monitors 114 and/or other system components can be maintained, serviced, replaced, and/or that provides new or replacement monitors 114 or other components. In the example of FIG. 1, the service and supply facility 130 is responsive to commands from the remote device 120, for example providing monitors 114 in response to an order request from the remote device 120. An example system includes dedicated monitors 114 for personnel, but any other arrangements are contemplated herein, such as utilizing a pool of monitors 114 that are checked out and/or utilized by operators, for example during a work shift.

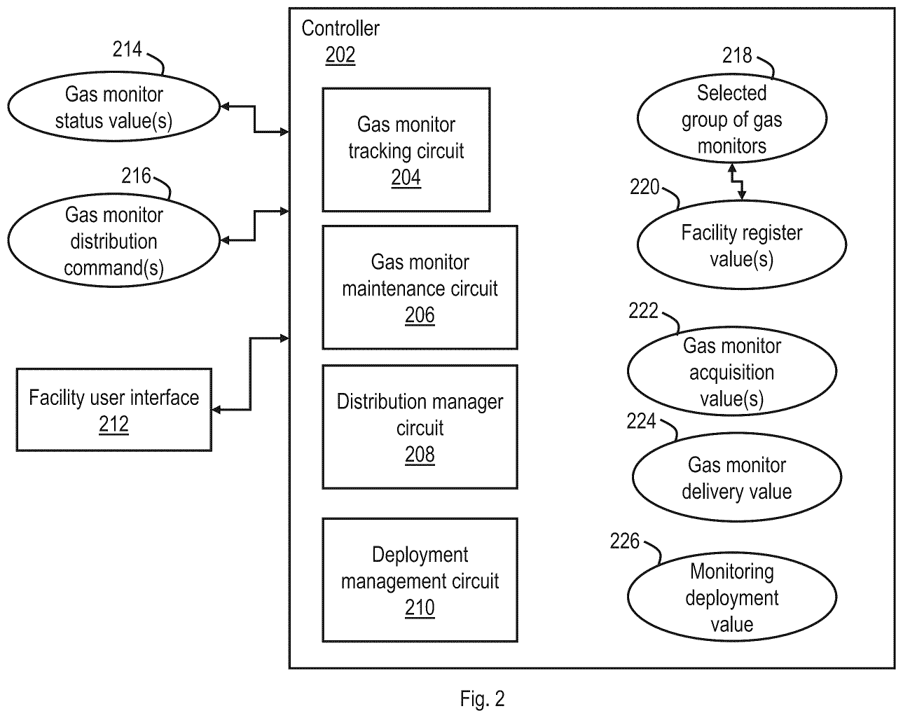

Referencing FIG. 2, an example controller 202 is depicted that supports operations to maintain, replace, and/or distribute monitors 114 to support gas monitoring at a facility 102. The example controller 202 may be a single device, for example positioned on one or more remote devices 120, 122, and/or may be a distributed device with aspects on any component of a system, for example on a data manager 116, gas monitor 114, bridging and/or extending monitors 124, 126, 128, and/or anchors 118. In certain embodiments, the components embodying the controller 202 may vary depending upon the operations being performed, the personnel interacting with the controller 202, and/or operating conditions of the related facility 102.

The example controller 202 includes a gas monitor tracking circuit 204 structured to interpret a gas monitor status value 214 for a selected group of gas monitors 218 (e.g., determining that one or more monitors of the group 218 are failed, have a fault, should be checked, are aging, and/or are no longer needed), and a gas monitor maintenance circuit 206 that determines a gas monitor acquisition value 222 (e.g., a list of gas monitors, including monitor type, sensing element characteristics, and/or other monitor capabilities) in response to the gas monitor status value 214. Additionally or alternatively, the gas monitor maintenance circuit 206 may determine the gas monitor maintenance circuit 206 may determine the gas monitor acquisition value 222 in response to determining that additional monitors are needed, including replacing monitors that are not completely functional (e.g., according to the gas monitor status value 214), and/or determining that additional monitors are needed (e.g., a team utilizing the group 218 now has additional members, needs additional monitors, and/or needs different monitors such as monitors having different sensing elements for a different type of gas and/or for a different range of gas concentrations, etc.). The example controller 202 further includes a distribution manager circuit 208 that provides a gas monitor distribution command 216 in response to the gas monitor acquisition value 222, for example providing an order to a SSF 130 to send monitors to the facility to replace monitors that are not fully functional and/or to provide additional monitors as needed at the facility. The operations of the controller 202 allow the facility 102 to seamlessly maintain the monitors needed for gas monitoring operations, respond to changes in the monitors needed, and to reduce downtime. In previously known systems, a responsible person would need to identify the need for replacement, additional, and/or alternative monitors, and then figure out how to order the replacements, and ensure the correct monitors are ordered, which may further require additional skill sets and thereby require multiple people and process handoffs to complete.

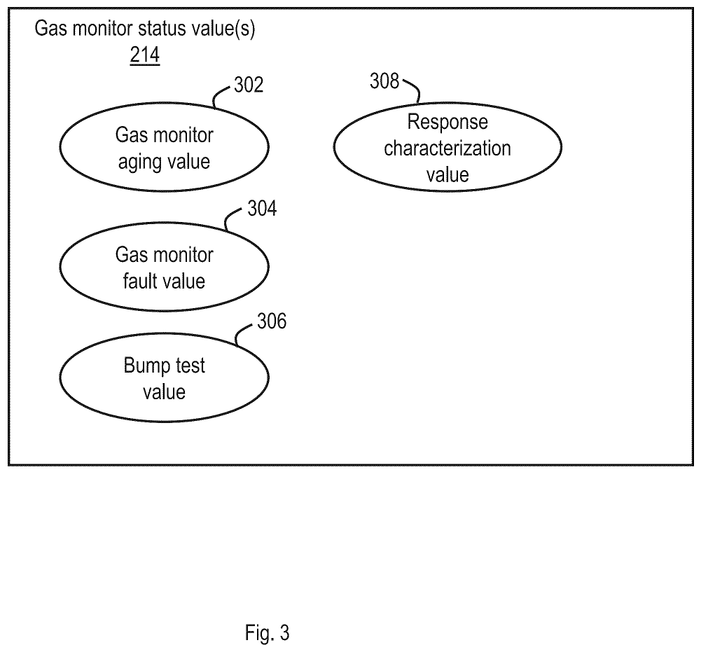

Referencing FIG. 3, example and non-limiting gas monitor status values 214 are schematically depicted. An example gas monitor status value 214 includes a gas monitor aging value 302—for example based on a calendar age of the gas monitor (e.g., used to replace gas monitors at a specified time period), and/or a utilization age of the gas monitor (e.g., based on operating hours, detected constituent throughput, a utilization aging index, or the like). An example gas monitor status value 214 includes a gas monitor fault value 304, for example a fault in the monitor, whether related to the sensing element, the power circuit, or any other aspect of the monitor that is capable to provide a digitally reportable fault that can be evaluated by the gas monitor tracking circuit 204. An example gas monitor status value 214 includes a bump test value 306, for example based on results of a bump test (e.g., ensuring the basic response of the gas monitor on exposure to a gas constituent, which may be the target gas constituent for the sensing element and/or a correlated gas constituent), or other rationality check for basic response of the gas monitor. An example gas monitor status value 214 includes a response characterization value 308 (e.g., a test to determine whether the gas monitor is responding correctly on exposure to a gas constituent, which may include any aspect such as dynamic response, rise/fall time, saturation response, and/or achieving the correct concentration determination within a specified time period). An example gas monitor status value 214 includes one or more aspects such as: a sensing element operating time, a sensing element throughput description, a sensing element version value (e.g., allowing for the replacement of older monitors), and/or a gas monitor firmware value (e.g., allowing for the replacement of monitors with older firmware versions, and/or highlighting monitors that should have a firmware update).

An example system includes the selected group of gas monitors 218 including a cooperative monitoring group associated with a facility (e.g., reference FIG. 1 and the related description). An example controller 202 is configured to maintain a specified number of gas monitors (and/or gas monitors of the correct type) for the cooperative monitoring group (e.g., maintaining 75 H₂S monitors for a facility).

An example gas monitor tracking circuit 204 interprets the selected group of gas monitors as a facility register value 220, wherein the gas monitor maintenance circuit 206 is further structured to determine a specified number of gas monitors 218 in response to the facility register value 220, and wherein the controller 202 is configured to maintain the specified number of gas monitors for the cooperative monitoring group. The facility register value 220 allows a user, for example a facility operator, supporting contractor, facility manager, safety personnel, or the like to maintain a register of the number and type of monitors utilized for the facility, where the gas monitor maintenance circuit 206 can ensure that the proper number of functional monitors are maintained for the facility. An example gas monitor tracking circuit 204 implements a facility user interface 212 (e.g., on a remote device 120, 122), and interprets the facility register value 220 in response to communications on the facility user interface 212 (e.g., providing a convenient method for the user to create, maintain, and/or update the register). An example gas monitor tracking circuit 204 interprets an updated facility register value 220, and updates the selected group of gas monitors 218 in response to the updated facility register value 220. An example controller 202 includes a distribution manager circuit 208 that implements a facility user interface 212, and provides the gas monitor distribution command 216 in response to communications on the facility user interface 212 (e.g., allowing a user to directly order a number and type of monitors).

An example controller 202 includes a deployment management circuit 210 that interprets a monitoring deployment value 226, where the gas monitor maintenance circuit 206 determines a gas monitor delivery value 224 in response to the monitoring deployment value 226, and where the distribution manager circuit 208 provides the gas monitor distribution command 216 in response to the gas monitor delivery value 224. The deployment management circuit 210 allows for an operator to conveniently order monitors for a specific deployment, for example as a temporary or continuing monitoring operation at the facility, and for the deployment monitors to be automatically maintained, without interfering with the main group of monitors for the facility (which may be separately maintained by the controller 202), for example allowing for rapid, temporary, and/or initial deployments of monitors at a facility.

An example monitoring deployment value 226 includes a description of a number and/or type of gas monitors. An example monitoring deployment value 226 includes a deployment duration value (e.g., after which the monitors for the deployment will be returned, converted to ordinary facility monitors, or otherwise resolved according to instructions in the monitoring deployment value 226). Example aspects of the type of gas monitor include a gas sensing type, a monitor communication capability (e.g., allowing for the specification of one or more broadcast capable monitors), and/or a monitor power capability (e.g., allowing for the specification of one or more monitors having an extended battery capability).

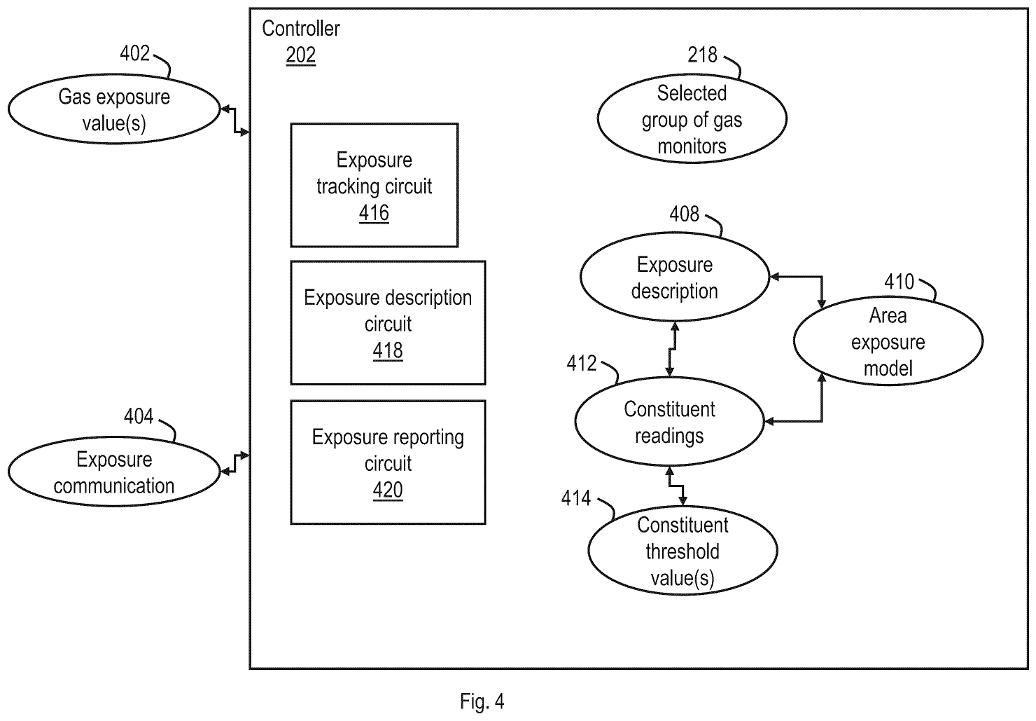

Referencing FIG. 4, an example controller 202 is depicted that supports tracking of worker or facility personnel exposure. The example controller 202 includes an exposure tracking circuit 416 structured to interpret gas exposure values 402 from a selected group of gas monitors 218 (e.g., monitors associated with a facility, a team, personnel of certain roles, and/or a particular area of a facility), an exposure description circuit 418 structured to determine an exposure description 408 in response to the gas exposure values 402, for at least one entity associated with the selected group of gas monitors 218. The at least one entity could be a facility, a team, personnel of certain roles, an area of the facility, and/or personnel associated with certain equipment and/or processes at the facility. The example controller 202 includes an exposure reporting circuit 420 structured to provide an exposure communication 404 in response to the exposure description 408. An example exposure description circuit 418 determines the exposure description in response to accumulated constituent readings from at least one of the selected group of gas monitors 218. In certain embodiments, the accumulated constituent readings may be non-linear, for example accumulating exposure values at a higher rate based on higher concentrations, and/or not accumulating exposure values below a threshold value (e.g., ignoring values below a threshold). Accordingly, the exposure description 408 may be based upon constituent readings 412 from the selected group of gas monitors 218, and/or a constituent threshold value 414 for exposure of the target constituent.

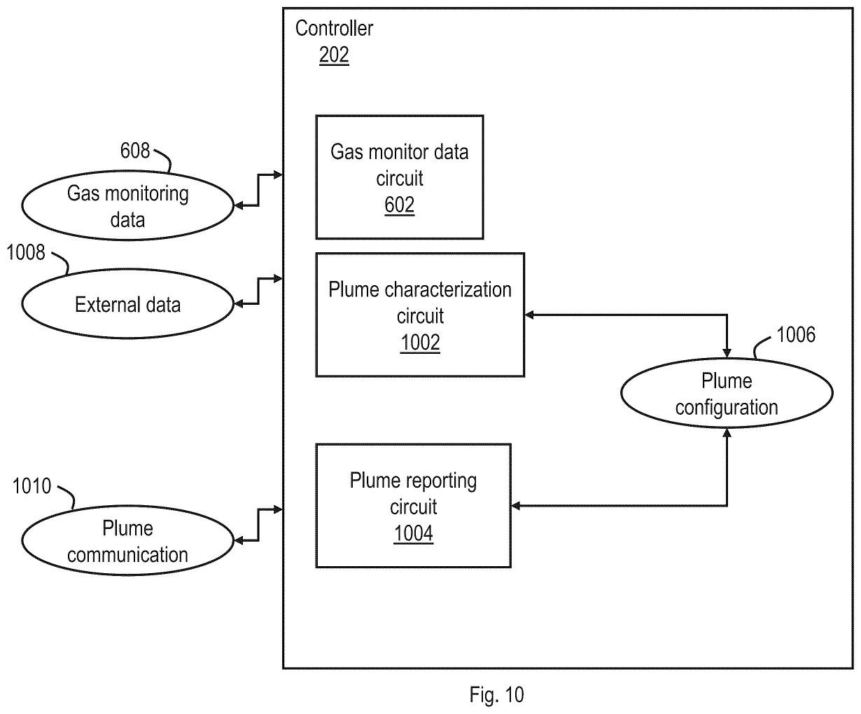

An example exposure description circuit 418 determines an area exposure model 410 in response to the accumulated constituent readings, for example allowing the estimation of exposure for personnel in an area that do not have an associated gas monitor, and/or that may have a gas monitor that is not functioning properly and/or does not measure the constituent of interest. In certain embodiments, operations to determine a plume configuration 1006 (reference FIG. 10 and the related description) may be utilized, at least in part, to determine the area exposure model 410. An example area exposure model 410 is determined in response to the gas exposure values 402.

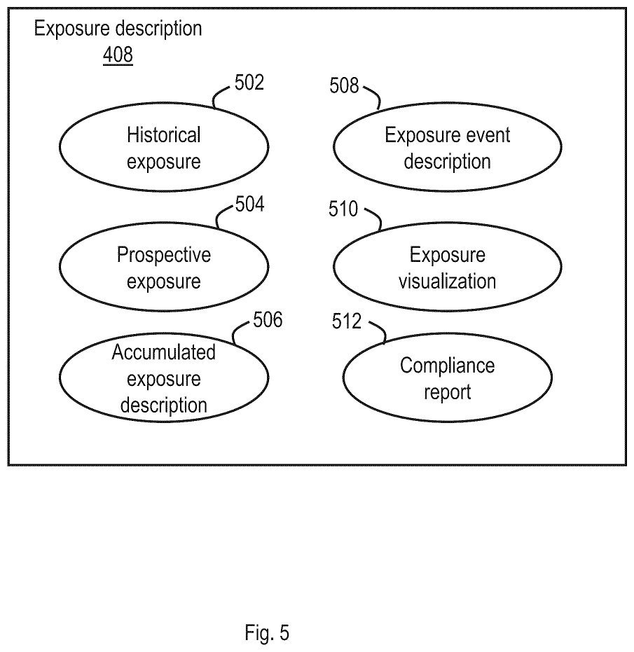

Referencing FIG. 5, example aspects of the exposure description 408 are schematically depicted. An example exposure description 408 is provided as a historical exposure 502 (e.g., determining and/or estimating actual exposure of the entity to the constituent of interest) and/or as a prospective exposure 504 (e.g., determining likely future exposure, for example to plan work shifts and rotations, to determine equipment upgrades, to plan future monitoring requirements, to plan evacuation pathing, and/or for impact assessment of events). An example exposure description 408 includes an accumulated exposure 506 description, where the accumulated exposure 506 is based on a selected time frame (e.g., a shift, a year, etc.), a lifetime exposure for the entity (e.g., a person), and/or any other time frame that may be relevant for regulatory purposes, health purposes, risk management purposes, according to industry standards, or the like. An example exposure description 408 includes an exposure event description 508 including data regarding aspects of the exposure event, including date, time, temperature, weather, wind speed, wind direction, precipitation, terrain, buildings/structures, source, gas composition, etc. Additionally or alternatively, an exposure description 408 includes a gas exposure of any level, for example where small accumulated exposures can create a risk and/or may be an indicator of a process issue for the facility that can be improved. In certain embodiments, an exposure description 408 includes confirmation that no significant exposure has occurred for a selected period of time, a selected facility location, and/or during selected facility operations (e.g., to confirm that a process and/or equipment is functioning properly, and/or to provide statistical information such as placing gas exposure events into an overall context and/or determining exposure rates). An example exposure description 408 includes an exposure visualization 510, for example a graph of exposure over time, a heat map of exposure (and/or exposure rates) for the facility or other organizing principle (e.g., a team, a job role, a particular shift or plant operation of the facility, etc.), and/or an impact visualization based on the exposure description 408 (e.g., based on costs and/or risks associated with the exposure). Where an exposure graph is utilized, the exposure graph may include a time coordinate and/or a location coordinate. An example exposure description 408 includes a compliance report 512, for example indicating compliance with a regulation, an industry standard, and/or a relevant policy for exposure based on the exposure description 408. The operations of the controller 202 of FIG. 4 allows a relevant user to readily determine whether the facility is compliant with personnel exposure requirements, to determine whether exposure issues have occurred and can assist in determining the causes of the occurrence (e.g., providing location information, facility operations information, relating exposures to time of day issues, etc.), determine whether future exposures are likely to occur based on the current facility parameters, and to take actions to mitigate and reduce exposure events.

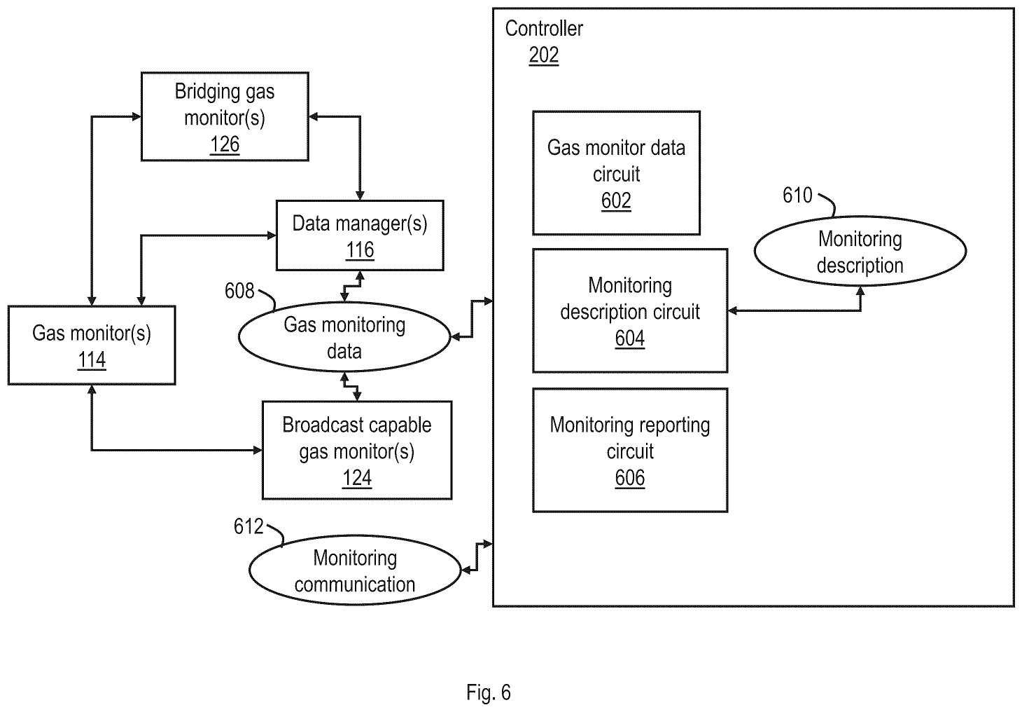

Referencing FIG. 6, an example controller 202 is depicted to support extending and bridging networks of gas monitors at a facility, ensuring that proper monitoring operations can be performed while reducing costs for infrastructure and device costs by enabling monitoring operations without using overdesigned monitoring devices with greater capabilities than are required to perform the monitoring operations. Further, the operations of the controller 202 of FIG. 6 support improved monitoring capability, reducing inconvenience to users and network gaps due to facility aspects such as network dead zones at the facility, improving overall safety for the facility and personnel associated therewith.

An example controller 202 includes a gas monitor data circuit 602 structured to interpret gas monitoring data 608 from at least one data manager end point 116 of a cooperative monitoring group 104 associated with a facility 102 for a first selected group of gas monitors 106, and from a broadcast-capable gas monitor 124, 128 for at least one additional gas monitor (e.g., 112 and/or 110, reference FIG. 1), wherein the first selected group of gas monitors 106 and the at least one additional gas monitor 112, 110 include gas monitor end points of the cooperative monitoring group 104. The broadcast-capable gas monitor 124, 128 includes at least one of a cellular transceiver or a satellite transceiver, allowing the broadcast-capable gas monitor 124, 128 to communicate directly with a remote device 120, 122, including on behalf of the at least one additional gas monitor 112, 110. The controller 202 includes a monitoring description circuit 604 that determines a monitoring description 610 in response to the gas monitoring data 608, and a monitoring reporting circuit 606 that provides a monitoring communication 612 in response to the monitoring description 610. Example and non-limiting monitoring communications 612 allow for relevant users to readily confirm monitoring operations and compliance, and ensure that monitors at the facility are operating properly, and confirm any events such as alarm events. An example controller 202 is embodied, at least in part, on a remote device 120, 122, including for example a device that is remote from the cooperative monitoring group 104, remote from the facility 102, and/or a cloud server. An example at least one additional gas monitor 112, 110 is communicatively coupled to the at least one data manager end point 116 via the broadcast-capable gas monitor 124, 128 (including potentially through the remote device 120, 122). An example system includes a mesh network having a first mesh portion including the first selected group of gas monitors 106, a second mesh portion including the at least one additional gas monitor 110, and wherein the broadcast-capable gas monitor 124 bridges the first mesh portion 106 and the second mesh portion 110. An example cooperative monitoring group 104 includes a mesh network, where the at least one additional gas monitor 110, 112 is communicatively coupled to the controller 202 via the broadcast-capable gas monitor 124, 128.

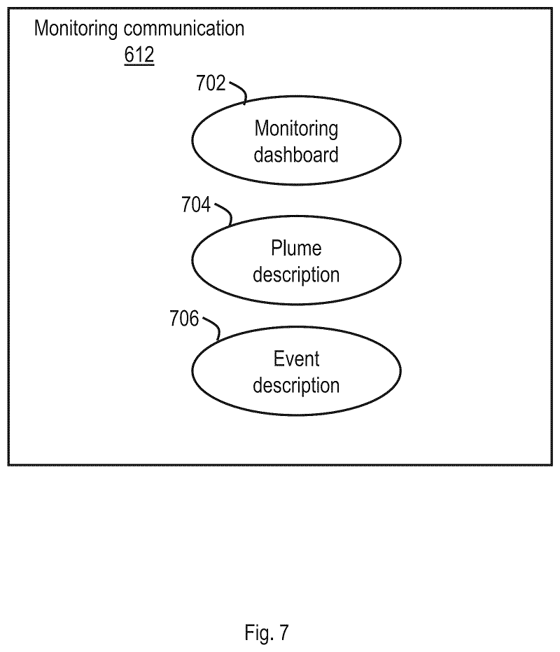

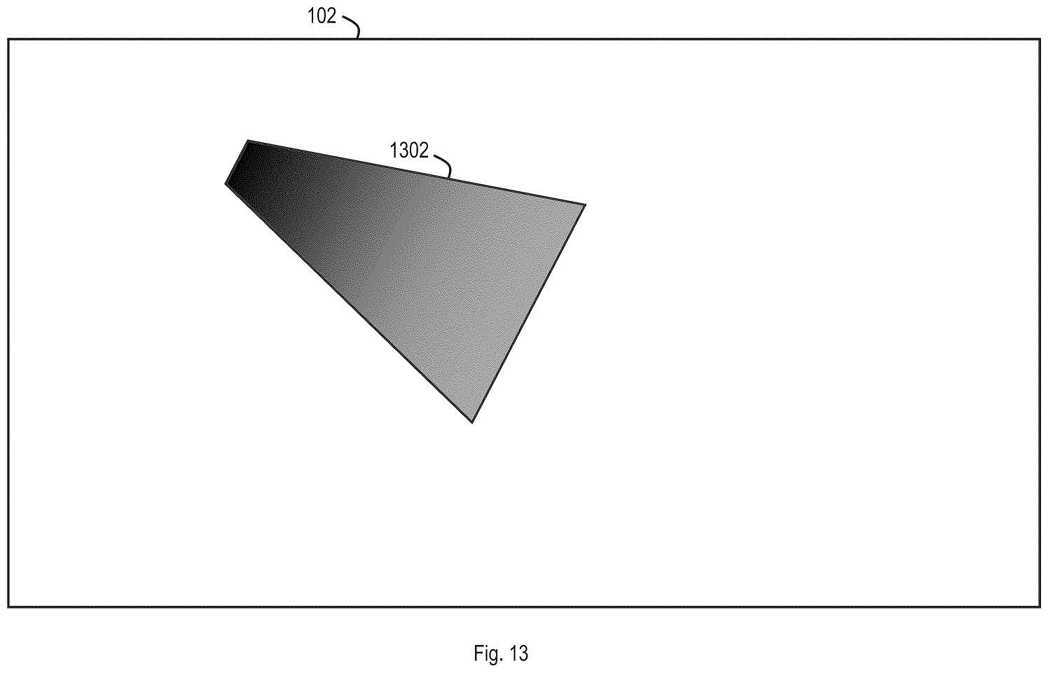

Referencing FIG. 7, example and non-limiting monitoring communications 612 are schematically depicted. An example monitoring communication 612 includes a monitoring dashboard 702, providing a relevant user with an overview of monitoring operations, highlights and/or notification of events (e.g., alarms, monitored gas values, facility heat maps of monitor compliance and/or gas values, monitor events such as test failures or fault conditions, etc.), a facility depiction with monitor locations and/or status, a facility depiction with current, past, or predicted gas values, data based on an area exposure model, facility register values, gas monitor acquisition values, and/or monitoring deployment values. In certain embodiments, the monitoring dashboard 702 can include any information available to the controller 202 and selected by the relevant user for display, allowing the user to configure the monitoring dashboard 702 for whatever information is important to that user, and/or related to gas monitoring and/or supporting operations to maintain gas monitoring at the facility. An example monitoring communication 612 includes a plume description, which may include a visualization of a relevant plume (e.g., reference FIGS. 13-15 and the relevant description), and/or any related parameters of interest (e.g., a highest value, a location of a highest concentration, a location of concentrations above a threshold, time values such as a plume start time and/or expected end time, peak plume time, etc.). An example monitoring communication 612 includes an event description 706, where the event can be related to any aspect of gas monitoring at the facility. Example and non-limiting events include one or more of: alarm events related to a monitor; a compliance event (including confirmation of compliance and/or notification of a non-compliant event); bump-test results and/or off-nominal bump-test occurrences; risk occurrences from any source; delivery and/or non-delivery of replacement, additional, and/or deployment monitors; loss-of-monitor events; loss-of-communication events (e.g., communication failures of a monitor, a bridging or extending monitor, an anchor, a gas station, etc.); firmware and/or calibration events (e.g., detection of outdated and/or non-compliant firmware and/or calibrations, and/or events related to updates or attempted updates of these for one or more monitors); and/or sub-alarm gas readings that are above a threshold value.

An example controller 202 includes a gas monitor data circuit 602 structured to interpret gas monitoring data 608 from at least one data manager end point 116 of a cooperative monitoring group 104 associated with a facility 102 for a first selected group of gas monitors 106, and communicatively couple the at least one data manager end point 116 with at least one additional gas monitor 108, 110, 112 via a bridging gas monitor 124, 126, 128, wherein the first selected group of gas monitors 106 and the at least one additional gas monitor 108, 110, 112 include gas monitor end points of the cooperative monitoring group 104, and wherein the bridging gas monitor 124, 126, 128 includes a battery configured to support independent operations of the bridging gas monitor 124, 126, 128 for at least a week. In certain embodiments, the bridging gas monitor 124, 126, 128 can readily be configured, in a portable format readily transportable by hand and less than about 8 kg in total weight, to operate independently for at least about 20 days. The example controller 202 includes a monitoring description circuit 604 structured to determine a monitoring description 610 in response to the gas monitoring data 608; and a monitoring reporting circuit 606 structured to provide a monitoring communication 612 in response to the monitoring description 610.

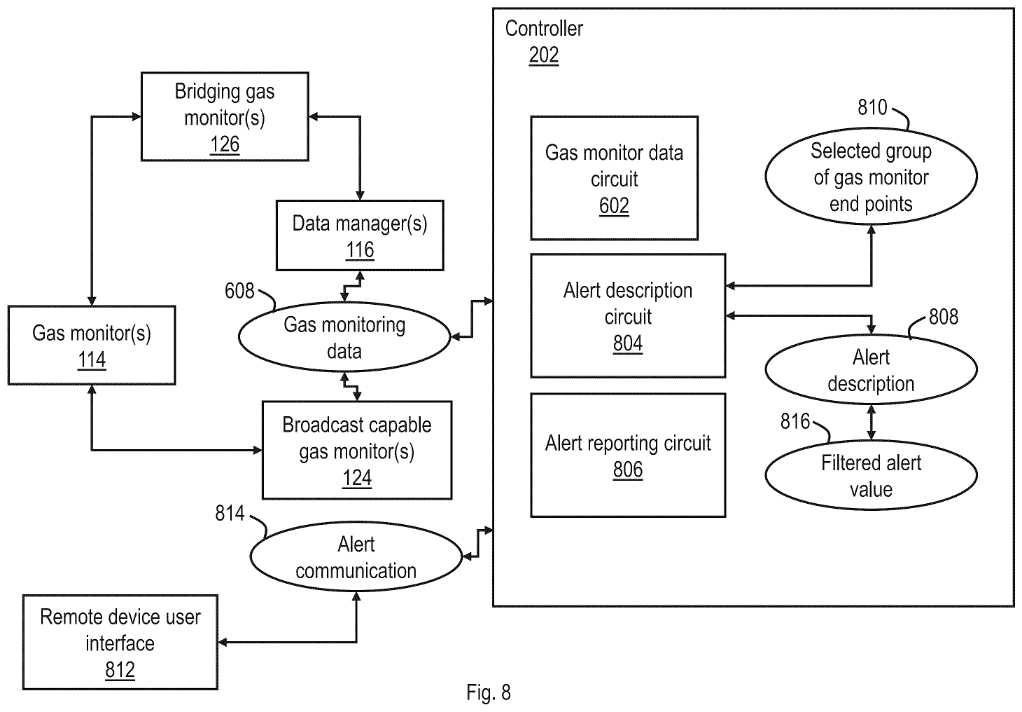

Referencing FIG. 8, an example controller 202 to perform alert operations related to gas monitoring at a facility is schematically depicted. Example alert operations include alert screening operations (e.g., ensuring that an alert is valid), promotion of alert notifications, and/or escalation of alert responses. An example controller 202 includes a gas monitor data circuit 602 structured to interpret gas monitoring data 608 from gas monitor end points 114 of a cooperative monitoring group 104 associated with a facility 102, an alert description circuit 804 structured to determine an alert description 808 in response to the gas monitoring data 608, and an alert reporting circuit 806 structured to provide an alert communication 814 in response to the alert description 808.

An example gas monitoring data 608 includes a gas monitor alert value (e.g., where the gas monitoring data 608 includes a gas monitor in an alert (or alarm) condition according to local readings at the gas monitor), where the alert description includes a filtered alert value 816. The filtered alert value 816 includes operations to confirm the gas monitor alert value, for example based on a persistence of the gas monitor alert value (e.g., the local alert at the gas monitor persists for a period of time), and/or a consistency of the gas monitor alert value (e.g., the readings from the alerting gas monitor appear stable and consistent, and/or are in agreement with nearby gas monitors, where available). The gas monitor alert value includes a local alert value (e.g., an indication of an alert by a specific gas monitor), and the alert communication 814 is provided to a user interface of a remote device 812. In certain embodiments, the filtered alert value 816 may be determined utilizing a quantitative filter (e.g., filter operations that adjust the value indicated by the alerting gas monitor), such as a low-pass filter of the gas monitor indicated concentration, using a moving average, using a delay period, or the like, to provide time to confirm that the alert value is real and/or sustained. In certain embodiments, the filtered alert value 816 may be determined using a qualitative or categorical filter (e.g., filter operations that do not adjust the value indicate by the alerting gas monitor, but instead adjust how that value is viewed for determining the alert description 808), for example determining that other related monitors are indicating gas concentrations that make sense in view of the alerting gas monitor, ensuring that the alerting gas monitor does not have faults, outdated operating parameters (e.g., old versions of calibrations, firmware, or sensing element hardware), improper operating parameters (e.g., incorrect settings for correlation algorithms, alerting thresholds, etc.), and/or other indications that the gas monitor may have an issue (e.g., deficient bump tests and/or sensing characterizations, or a lack of recent bump tests, indicators of degradation such as a trajectory of tests or characterizations that have been moving toward non-compliance, recent changes in sensing performance, etc.). While the filtered alert value 816 is utilized to ensure that a local gas monitor alert is valid, in certain embodiments operations of the controller 202 and/or the system generally will not disturb the local gas monitor alert (e.g., the operator associated with the gas monitor will get an alert and would take expected actions such as leaving the area and/or donning appropriate PPE for the alert event). Further, the controller 202 in certain embodiments will continue to show the presence of the local alert, for example on a monitoring dashboard 702 and/or in a log file associated with gas monitoring data. In certain embodiments, the filtered alert value 816 is utilized to diagnose the event and/or the related gas monitor, to flag that it is possible that a particular alert is not a real alert, utilized in modeling based on the gas monitor data (e.g., plume modeling and/or in an area exposure model), utilized in future design and construction of gas monitors, utilized in calibration settings for sensing elements of gas monitors, utilized in investigation and/or reporting of the event (including, for example, reporting of both the local alert and the determination of whether the alert was a real alert reflecting an actual gas concentration event), and/or utilized in determining a broader event response (e.g., determining the scope of broader alert messages to other gas monitors in the area, determining evacuation scope, determining the scope of contact and information to be provided to emergency responders, etc.).

The operations of the controller 202 as set forth in FIG. 8 are described in the context of determining that an indicated local alert may not be a real gas concentration event. In certain embodiments, the controller 202 is utilized to determine that a gas monitor is not providing a local alert when it should be, although such operations will occur less frequently for several reasons, including at least: gas monitor alerts tend to be set conservatively (e.g., to protect users of the gas monitors); the gas monitor in a “not alert” status will not activate, so the controller 202 may not have a stimulus to perform the consistency check; certain operations to determine a missed alert, such as determining a plume model, may take time and may be determined after the operator associated with the gas monitor has left the alert location; and an alerting gas monitor will generally lead to personnel leaving the area, so it is not as likely that any given gas monitor is the first one to detect a gas concentration event, which will tend to drive other personnel (with their associated gas monitors) out of the area before the gas monitor is significantly exposed to the gas concentration event. Nevertheless, the controller 202 in certain embodiments will have the information available to determine that a gas monitor has not alerted when it should have done so (a “suspect gas monitor”, following), for example when a plume configuration 1006 (reference FIG. 10 and the related description) determines that a gas monitor was present in a significant gas concentration, the controller 202 can determine the indicated gas concentration of that gas monitor at the time it was within the significant gas concentration, and operate as described to determine that the gas monitor had a “not alert” status in the context of a real gas concentration event. In such an event, the operations of the controller 202 are the same, where the gas monitoring data 608 from the suspect gas monitor indicates no alert, and where the operations of the alert description circuit 804 (e.g., based on surrounding gas monitors, and/or based on a plume configuration 1006 as set forth in FIG. 10 and the related description) determine an alert description 808 indicating that the suspect gas monitor should have been in alert. In the example, the alert reporting circuit 806 provides alert communications 814 in the same manner as described for an alert that was not estimated to be a real event, which may include providing a message to the suspect gas monitor (e.g., warning the operator to move to a safe location) if the alert estimation is still relevant (e.g., if the gas monitor is still in a position consistent with the alert description 808 estimation of the real gas concentration). Further, the alert reporting circuit 806 can provide alert communications 814 to the relevant personnel with the data utilized to determine the missed alert, whether determined at a time when the alert should have been active or later (e.g., in a post-processing event), including indications that the suspect gas monitor should be checked, and/or removing the suspect gas monitor from service.

In some embodiments, the alert description circuit 804 is further structured to determine the alert description 808 in response to a plume model analysis. The alert description circuit 804 is further configured to determine a selected group of the gas monitor end points 810 in response to the plume model analysis (e.g., based on gas monitors that are affected by the plume, and/or that may be affected by the evolving plume, where affected by includes potential exposure events, but may include gas monitors associated with event response such as to team members of individuals with gas monitors affected by the plume or may be affected by the evolving plume, managers, supervisors, incident response team members, or the like), and wherein the alert communication 814 includes a notification provided to the selected group of the gas monitor end points 810. The alert description circuit 804 is further configured to determine the selected group of the gas monitor end points 810 in response to a location value for the gas monitor end points.

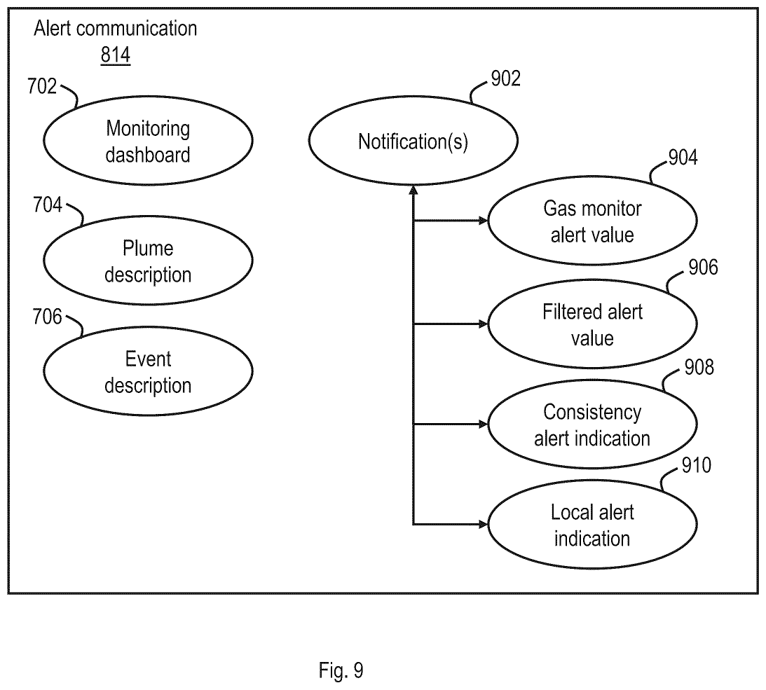

Referencing FIG. 9, example and non-limiting alert communications 814 are schematically depicted. An example alert communication 814 includes a monitoring dashboard 702 communication, for example providing a notification on a monitoring dashboard of the local alarm and/or the alert description 808 (e.g., including a confirmation of the local alarm, and/or a notification that the local alert may not be a real gas concentration event). An example alert communication 814 includes a plume description 704, for example providing a plume visualization or other data in view of the alert description 808, for example providing a plume depiction based on the local alert being real or not real (e.g., allowing the user to see both the plume as estimated based on the real local alert and as estimated if the local alert is not real; allowing the user to select between these, and/or showing one or the other with a notification that potentially conflicting data is indicated). An example alert communication 814 includes an event description 706, for example indicating the local alert event, the potential that the local alert event is not real (if present, based on the alert description 808), and/or indicating the event if the local alert event is not real (e.g., the relevant gas monitor is still likely to have an issue that should be investigated, even if the local alert event is not a real gas concentration event). The utilization of the alert communication 814 by the controller 202 enhances the facility operations and safety by allowing the relevant user to more rapidly determine the root cause of events, allows the relevant user to identify and correct gas monitor issues more quickly, reduces operator fatigue in broad responses to events that are not real gas concentration events, and ensures that event evaluation is more likely to address the true cause of events rather than responding to the wrong issues for events. In certain embodiments, for example where a gas monitor provides one or more local alerts that are not real, not confirmed, and/or suspect, operations of the controller 202 may automatically take the gas monitor out of service and/or replace the gas monitor (e.g., reference FIG. 2 and the related description), reducing any potential downtime due to issues with the gas monitor and improving event response by eliminating potential confounding issues related to the event.

An example alert communication 814 includes a notification 902, which may be provided to any relevant user and/or device in the system, for example to safety personnel, management, supervisors, support personnel for the gas monitoring system, or the like. The notifications 902 provided to different users may depend upon the role of the user, and may be set according to a plan for the facility, including considerations for the applicable regulatory, compliance, policy, and/or industry standard environment for the facility and/or the particular gas constituent relevant to the gas monitoring and/or local alert. Example notifications 902 may be provided to any user interface, email, web portal, mobile application, proprietary application in communication with the system (e.g., utilizing an API to interact with the controller 202), text message, device message (e.g., to a gas monitor and/or other end point of the cooperative monitoring group 104), etc. Example notifications 902 include one or more aspects such as: a gas monitor alert value 904 (e.g., the estimated alert value as indicated by the alert description 808); a filtered alert value 906 (e.g., the determined filtered alert value, including potentially underlying information such as the quantitative and/or qualitative aspects utilized to determine the filtered alert value, for example helping a relevant user understand any differences between the local alert and the alert description 808, and which may include a depiction of nearby gas monitors and indicated readings from those monitors); a consistency alert indication 908 (e.g., indicating that an inconsistency is present based on the output of the alerting gas monitor, based on the output of nearby gas monitors, and/or on a difference between the gas monitor alert value 904 and the local alert value); and/or a local alert indication 910 (e.g., allowing the user to see the local alert, even if it is not estimated to be a real gas concentration event).

In an example embodiment, the gas monitoring data 608 includes a gas monitor alert value includes a local alert for a first gas monitor end point, and wherein the alert description circuit 804 is further structured to determine the alert description 808 in response to gas monitor data 608 indicating a local alert (or not) for at least one additional gas monitor end point. An example alert description circuit 804 determines the alert description 808 in response to a positional relationship between the first gas monitor end point and the at least one additional gas monitor end point (e.g., determining based on how close or far the monitors are, and/or based on the alert values for surrounding gas monitors). An example alert description circuit 804 determines the alert description in response to a fluid relationship between the first gas monitor end point and the at least one additional gas monitor end point (e.g., monitors that are close to each other but fluidly separated are not likely to share ambient gas concentrations, while monitors that may be distant but have a strong fluid connection, such as a connecting hallway, a prevailing wind or air movement connection, and/or that are thermally connected such as vertically displaced air having a thermal gradient may be likely to share ambient gas concentrations, including potentially with a estimable time delay factor). An example alert description circuit 804 determines the alert description 808 in response to a plume model analysis (e.g., reference FIG. 10 and the related description). In certain embodiments, the alert communication 814 includes a notification 902 to gas monitors withing the comparison group based on the plume model analysis (e.g., warning users where they are in an area that is estimated to have a gas concentration event, and/or an imminent gas concentration event). The comparison group may include gas monitors that are in a local proximity to the gas concentration event, to other alerting monitors, and/or that are in a fluid connection proximity to these.

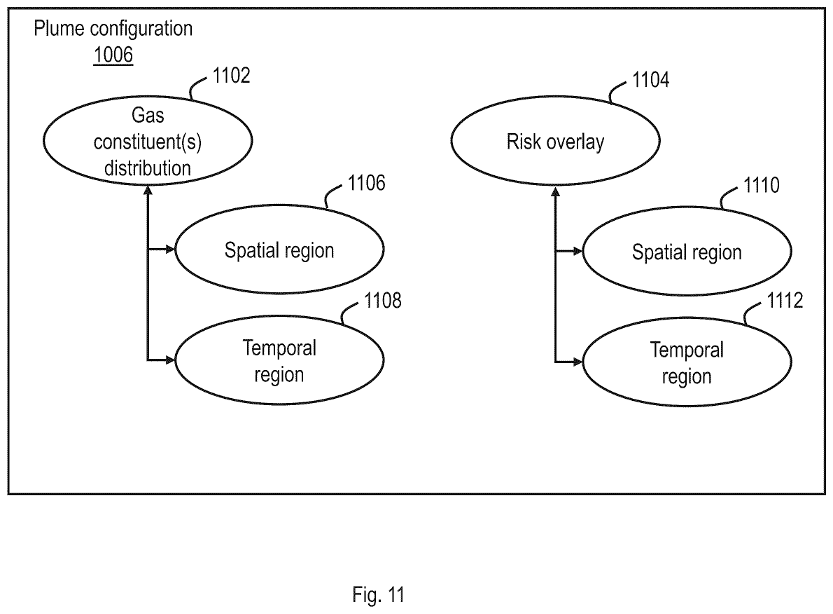

Referencing FIG. 10, an example controller 202 configured to perform plume modeling and to provide plume communications 1010 is schematically depicted. The example controller 202 includes a gas monitor data circuit 602 structured to interpret gas monitoring data 608 from gas monitor end points of a cooperative monitoring group 104 associated with a facility 102, and a plume characterization circuit 1002 structured to determine a plume configuration 1006 including a gas constituent distribution 1102 (reference FIG. 11) in response to the gas monitoring data 608. The controller 202 includes a plume reporting circuit 1004 structured to provide a plume communication 1010 in response to the plume configuration 1006.

The plume configuration 1006 includes a description of the plume that can be utilized to provide graphical depictions of the plume, notifications related to the plume, and to make determinations relevant to the plume such as the estimated gas concentration that is estimated to be present at gas monitors in the system, including relating the estimated gas concentration to the time and position of the gas monitor. The plume itself may be a gas concentration based plume, for example the gas concentration of a gas constituent of interest, and/or the plume may be a risk plume (e.g., an area of increased risk based on gas concentrations estimated to be in the area). For example, a given gas constituent at a given concentration may have a different risk profile based on many factors, such as the number of personnel in an area, whether personnel in the area have appropriate PPE and/or are expecting to be working in an area with the gas constituent of interest, the operating conditions of the facility (e.g., the temperatures, pressures, vibration profile, material composition of machinery, composition of any gases, working fluids, or other product materials in the area, etc.), and/or conditions in the region surrounding the facility where applicable. The risk profile may accordingly be non-linear with respect to the gas constituent concentration, and may vary according to external factors such as time of day and facility operations. Further, while plume modeling operations herein utilize gas concentration estimates for gas constituents of interest through a selected spatial region (e.g., physical or geographic location and distribution of the concentrations) and/or through a selected temporal region (e.g., an absolute time such as a calendar time and date, or a relative time such as a time since an event occurs, and which may include a time frame of interest such as a period of minutes, hours, or days), the risk profile may have different impacts based on the accuracy of the gas concentration estimate, and accordingly the resolution of the gas concentration estimate that provides for a useful plume configuration 1006 can vary significantly, so the parameters utilize to estimate the plume may similarly vary significantly depending upon the overall situation and purpose of the plume estimate. For example, a plume estimator for a relatively inert macro gas that is only a risk at significant concentrations, for example where the gas constituent of interest is nitrogen, carbon dioxide, or water (humidity), a relatively coarse estimate of the gas concentration may be sufficient to provide a useful plume estimate. Similarly, some gases that are a high risk at very low concentrations (e.g., H₂S, CN) may also provide a useful plume estimate even where the plume estimator provides a coarse estimate, as the presence of such gases in any significant concentration is a significant risk to human life and health, and a high resolution model of the evolving concentration may not add significantly to the utility of the plume model. By contrast, some gases that have significant concentration thresholds where the risk increases non-linearly (e.g., explosive gases that are not immediately dangerous at low concentrations but can reach a lower explosive limit (LEL), such as low carbon count hydrocarbons), and/or where the risk accumulates with continued exposure and thus integrating low concentrations over time informs the risk analysis (e.g., carbon monoxide), may significantly benefit the risk management with a relatively high resolution plume estimate. Further, the purpose of utilizing the plume estimate can inform the relevant resolution of the plume model that is beneficial. For example, determining evacuation timing and routing may not indicate a high resolution model, but utilizing the plume model to determine whether a gas monitor is correctly reading a gas concentration and/or whether the gas monitor should have alerted (or not) may indicate a relatively high resolution model (at least at the concentrations relevant to the gas monitor sensing element). Accordingly, a number of parameters and techniques for determining the plume configuration 1006 are set forth herein, a number of which are useful in certain embodiments, at certain operating conditions, and for certain purposes, and some of which are not needed for other embodiments, operating conditions, and/or purposes. One of skill in the art, having the benefit of the present disclosure, can readily determine the parameters and operations to configure the plume characterization circuit 1002 to determine the plume configuration 1006 as set forth herein, having knowledge generally available when contemplating a particular gas monitoring system for a particular facility. Certain considerations for configuring the plume characterization circuit 1002 to determine the plume configuration 1006 include, without limitation: the gas constituents utilized at, and/or which may be generated (e.g., including intentional generation, and/or generation of certain gases as a failure mode of operations of the facility such as the mixing of products at the facility that are not designed to be mixed, but where such may occur in response to an equipment or process failure); the pressures and/or temperatures of operating equipment, process streams, and/or the ambient air at the facility; the variability of processes at the facility, including regular variability (e.g., operations by shifts and/or over a scheduled time period) and/or ad hoc variability (e.g., changes made to meet varying customer demand, to respond to varying product input compositions, etc.); the number of personnel at the facility in proximity to risk areas, including the training of such personnel and the availability and/or utilization of PPE relevant to gas constituents of interest; the distribution of gas monitors at the facility, including numbers, positioning, and the trajectory in time and/or space of these during operations; the detection ranges and/or resolution of gas monitors at the facility, including the relationship of these with the concentration of the gas constituents of interest and/or other factors (e.g., ambient temperature, humidity, barometric pressure, etc.); the presence and/or quantization of sensitive areas surrounding the facility, and/or of buffer zones around the facility (e.g., a facility that is five miles from any other facility or buildings, compared to a facility adjacent to a residential area); the prevailing winds and/or airflow within the facility and/or in the environment of the facility; the risk mitigation scheme of the facility, including utilization and/or acceptability of evacuation, managing countermeasures (e.g., flooding or misting areas, flushing with compressed air, etc.), equipment and process hardening (e.g., to reduce the likelihood of failures), and/or PPE for personnel at the facility; and/or regulations, policies, and/or industry standards relevant to the facility and processes performed at the facility.

An example plume configuration 1006 includes the gas constituent distribution over a selected spatial region. The gas constituent distribution may be determined according to gas constituent readings at gas monitors distributed at the facility, including estimating a gas constituent distribution that is consistent with the available gas monitor readings. The gas constituent distribution may further include estimation of a likely source point of the gas constituent, a release model that evolves over time, and/or a release progression that results in the observed constituent distribution. The gas constituent distribution may further be determined in response to prevailing air movement in the facility and/or according to the wind (e.g., for locations that are outdoors and/or affected by outdoor air movement), for example when estimating how the present distribution will evolve going forward, and/or how the present distribution has evolved from a likely release event. The gas constituent distribution may further be determined in response to the fluid connectivity of portions of the facility, and temperatures in the facility (e.g., to account for air layering and/or likely vertical movement of the air). The gas constituent distribution may further be determined in response to the molecular weight of the gas constituent of interest and/or the overall air composition (e.g., to model settling and/or diffusion). The gas constituent distribution may involve both feedforward and feedback elements, for example predicting how the gas constituent will distribute, and then confirming the prediction and/or adjusting the estimate in response to actual feedback from gas monitors that remain at the scene. The gas constituent distribution may account for the confidence value of particular gas monitors, for example discounting the input of a suspect gas monitor to the model, and enhancing the input of a highly trusted gas monitor to the model.

An example plume configuration 1006 includes the gas constituent distribution over a selected temporal region. The time estimated may be a future time (e.g., where is the plume going over the next hour), or a past time (e.g., based on an estimated source, where did it go between then and a future time, such as the present). In certain embodiments, the plume model may be utilized to verify that the facility design worked according to a plan (e.g., did the gas evolve as it was planned to in the event of an incident), to verify proper operation of gas monitors (e.g., did the gas monitors within the plume provide the expected results, in a further example if one gas monitor is not consistent then that gas monitor may be the problem, and if several gas monitors are not consistent then the model may be tuned to match the observation, and/or gas monitors involved may be tested after the incident to determine how they should be viewed against the model).

In certain embodiments, the plume configuration 1006 includes a risk overlay rather than a direct gas constituent distribution. In certain embodiments the risk overlay is determined in time and/or space as with the gas constituent distribution, where the risk is determined in response to the gas constituent concentration. As described preceding, the risk determination may be non-linear, and can follow any scheme such as saturation (e.g., anything over 10 ppm for the constituent of interest is a “maximum risk” value), reversals, hysteresis (e.g., to account for uncertainty and/or to avoid dithering of risk values), or the like. In certain embodiments, the risk overlay may be adjusted for external factors, such as active processes at the facility, time of day, calendar date (e.g., weekends or holidays may have a distinct risk-concentration relationship compared to normal working weekdays), current weather conditions, or the like.

The example plume communication 1010 can include any type of communication in the system that is accessible to the controller 202. Example and non-limiting plume communications include one or more communications such as: a visual display of the concentration and/or risk plume, plotted in space and/or time; a communication of a suspect gas monitor for evaluation and/or replacement; a communication to a gas monitor in response to the plume, including an alert, an evacuation order, a wellness check, and/or an evacuation routing communication; a communication to a remote device, including to a safety response team, manager, supervisor, system administrator, gas monitoring support personnel, and/or regulatory personnel; and/or a communication of gas monitor values and/or plume modeling values, and/or an instruction to save one or more of these for post-processing.

Referencing FIG. 11, example and non-limiting plume configurations 1006 include one or more of: a gas constituent distribution 1102; a spatial gas constituent distribution 1106; a temporal gas constituent distribution 1108; a risk overlay 1104; a spatial risk overlay 1110; and/or a temporal risk overlay 1112.

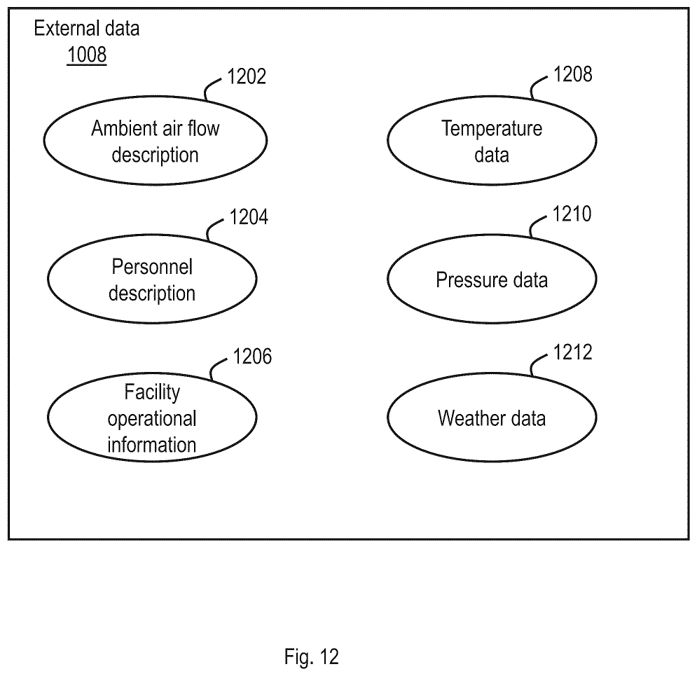

Referencing FIG. 12, example external data 1008 that may be utilized to determine the plume configuration include, without limitation: an ambient air flow description 1202; a personnel description 1204 (e.g., numbers, locations, roles, evacuation routes and gathering points, etc.); facility operational information 1206 (e.g., processes and parameters therefore, and/or any facility operation information as set forth throughout the present disclosure); temperature data 1208 (e.g., ambient temperature, facility temperature, equipment temperature, process stream temperatures, etc.); pressure data 1210 (e.g., ambient pressure, facility pressure, process stream pressure, etc.); and/or weather data 1212 (e.g., temperatures, winds, precipitation, and/or the outlook for these over the relevant time frame).

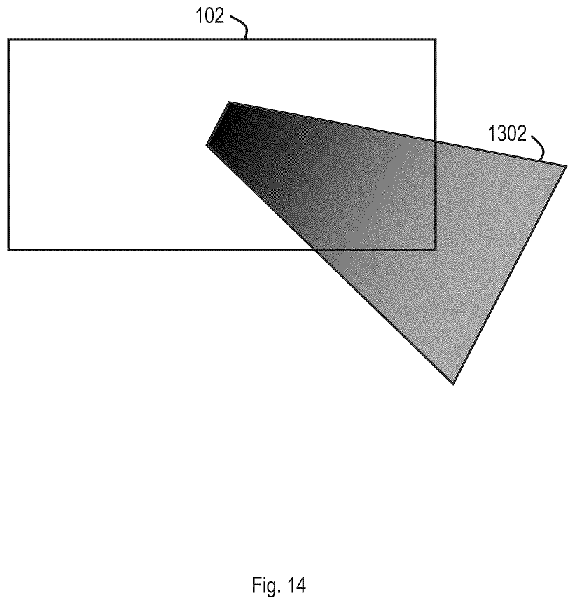

Referencing FIG. 13, an example overlay 1302 according to an illustrative plume configuration 1006 is depicted, showing an estimated concentration and/or risk plume at a facility 102. Referencing FIG. 14, an example overlay 1302 is depicted, making it clear in the example of FIG. 14 that the plume and/or overlay is not limited to the facility 102.

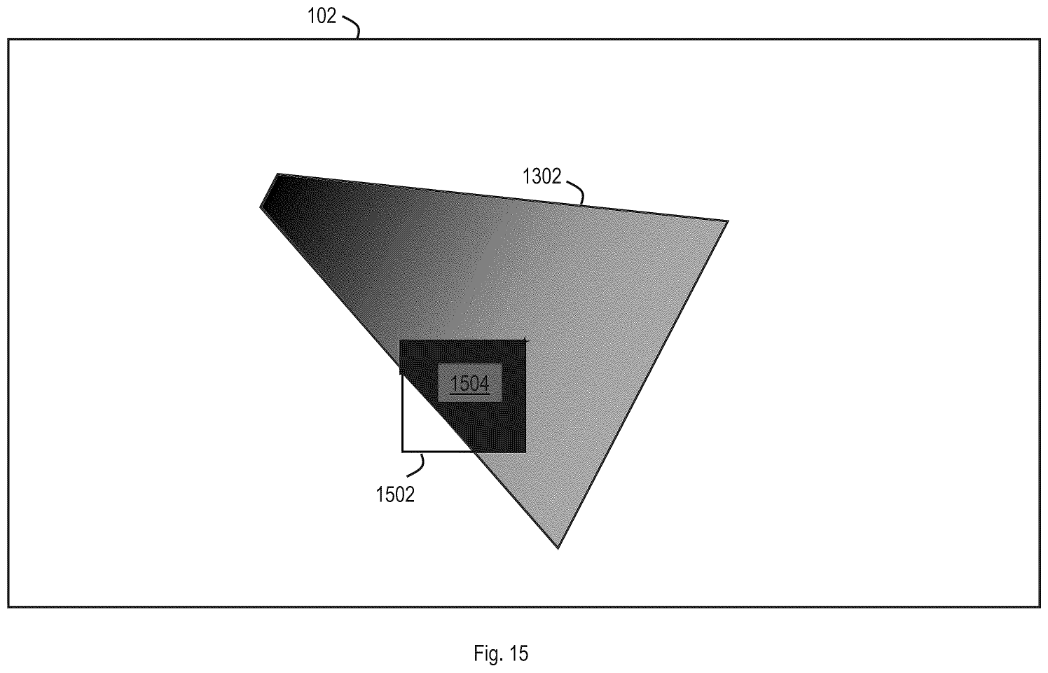

Referencing FIG. 15, an example overlay 1302 is depicted, for example with an area 1502 (which includes area portion 1504 that overlaps with the plume) within the facility where the risk is higher due to interaction with the plume (e.g., where a process is occurring and/or personnel are located that have an enhanced risk or sensitivity to the gas constituent(s) represented by the plume).

An example procedure for supporting gas monitor distribution support is described following. The example procedure may be performed by any system, apparatus, controller, circuit, or other components as set forth throughout the present disclosure, including without limitation a controller 202 such as depicted and described in reference to FIG. 2.

The example procedure includes an operation to interpret a gas monitor status value for a selected group of gas monitors, an operation to determine a gas monitor acquisition value in response to the gas monitor status value, and an operation to provide a gas monitor distribution command in response to the gas monitor acquisition value. Certain further operations of an example procedure are described following, any one or more of which may be performed with the example procedure. An example operation includes maintaining a specified number of gas monitors for a cooperative monitoring group associated with a facility, wherein the cooperative monitoring group comprises the selected group of gas monitors. An example operation includes interpreting the selected group of gas monitors as a facility register value, determining a specified number of gas monitors in response to the facility register value, and maintaining the specified number of gas monitors for a cooperative monitoring group associated with a facility, wherein the cooperative monitoring group comprises the selected group of gas monitors. An example operation includes implementing a facility user interface, and interpreting the facility register value in response to communications on the facility user interface. An example operation includes interpreting an updated facility register value in response to communications on the facility user interface, and updating the selected group of gas monitors in response to the updated facility register value. An example operation includes interpreting a monitoring deployment value, determining a gas monitor delivery value in response to the monitoring deployment value, and providing the gas monitor distribution command further in response to the monitoring deployment value.

An example procedure for tracking worker exposure is described following. The example procedure may be performed by any system, apparatus, controller, circuit, or other components as set forth throughout the present disclosure, including without limitation a controller 202 such as depicted and described in reference to FIG. 4.

The example procedure includes an operation to interpret gas exposure values from a selected group of gas monitors, an operation to determine an exposure description, in response to the gas exposure values, for at least one entity associated with the selected group of gas monitors, and an operation to provide an exposure communication in response to the exposure description. Certain further operations of an example procedure are described following, any one or more of which may be performed with the example procedure. An example operation includes determining the exposure description in response to accumulated constituent readings from at least one of the selected group of gas monitors. An example operation includes wherein the at least one entity comprises a facility location, and determining an area exposure model in response to the accumulated constituent readings. An example operation includes determining the exposure description as at least one of a historical exposure or a prospective exposure. An example operation includes determining the exposure description in response to a constituent threshold value. An example operation includes determining an area exposure model in response to the gas exposure values, and determining the exposure description further in response to the area exposure model. An example operation includes determining the exposure description as at least one of a historical exposure or a prospective exposure.

An example procedure for bridging and/or extending mesh networks, including for a cooperative monitoring group of gas monitors for a facility, is described following. The example procedure may be performed by any system, apparatus, controller, circuit, or other components as set forth throughout the present disclosure, including without limitation a controller 202 such as depicted and described in reference to FIG. 6.

The example procedure includes an operation to interpret gas monitoring data from at least one data manager end point of a cooperative monitoring group associated with a facility for a first selected group of gas monitors, an operation to interpret gas monitoring data from a broadcast-capable gas monitor for at least one additional gas monitor, wherein the first selected group of gas monitors and the at least one additional gas monitor comprise gas monitor end points of the cooperative monitoring group, an operation to determine a monitoring description in response to the gas monitoring data, and an operation to provide providing a monitoring communication in response to the monitoring description. Certain further operations of an example procedure are described following, any one or more of which may be performed with the example procedure. An example operation includes, wherein the cooperative monitoring group comprises a mesh network comprising a first mesh portion including the first selected group of gas monitors and a second mesh portion including the at least one additional gas monitor, and bridging the first mesh portion and the second mesh portion utilizing a broadcast-capable gas monitor.

Another example procedure includes an operation to interpret gas monitoring data from at least one data manager end point of a cooperative monitoring group associated with a facility for a first selected group of gas monitors, an operation to communicatively couple the at least one data manager end point with at least one additional gas monitor utilizing a bridging gas monitor, an operation to determine a monitoring description in response to the gas monitoring data, and an operation to provide a monitoring communication in response to the monitoring description.

An example procedure for providing alert communications for a cooperative monitoring group of gas monitors for a facility, is described following. The example procedure may be performed by any system, apparatus, controller, circuit, or other components as set forth throughout the present disclosure, including without limitation a controller 202 such as depicted and described in reference to FIG. 8.

The example procedure includes an operation to interpret gas monitoring data from gas monitor end points of a cooperative monitoring group associated with a facility, an operation to determine an alert description in response to the gas monitoring data, and an operation to provide an alert communication in response to the alert description. Certain further operations of an example procedure are described following, any one or more of which may be performed with the example procedure. An example operation includes, wherein the gas monitoring data comprises a gas monitor alert value, and wherein the alert description comprises a filtered alert value, and determining the filtered alert value in response to a persistence of the gas monitor alert value. An example operation includes, wherein the gas monitoring data comprises a gas monitor alert value, and wherein the alert description comprises a filtered alert value, and determining the filtered alert value in response to a consistency of the gas monitor alert value. An example operation includes, wherein the gas monitoring data comprises a gas monitor alert value, and wherein the alert description comprises a filtered alert value, and determining the alert description in response to at least one further gas monitor alert value for at least one additional gas monitor end point. An example operation includes determining the alert description in response to a positional relationship between a gas monitor end point of the gas monitor end points of the cooperative monitoring group and the at least one additional gas monitor end point. An example operation includes determining the alert description in response to a fluid relationship between a gas monitor end point of the gas monitor end points of the cooperative monitoring group and the at least one additional gas monitor end point. An example operation includes determining the alert description in response to a plume model analysis. An example operation includes determining a selected group of the gas monitor end points in response to the plume model analysis, and wherein providing the alert communication comprises providing a notification to the selected group of the gas monitor end points. An example operation includes determining the selected group of the gas monitor end points in response to a location value for the gas monitor end points. An example operation includes wherein providing the alert description comprises providing a notification on a monitoring dashboard implemented on a remote device.

An example procedure for providing plume modeling and visualization for a cooperative monitoring group of gas monitors for a facility, is described following. The example procedure may be performed by any system, apparatus, controller, circuit, or other components as set forth throughout the present disclosure, including without limitation a controller 202 such as depicted and described in reference to FIG. 10.

An example procedure includes an operation to interpret gas monitoring data from gas monitor end points of a cooperative monitoring group associated with a facility, determining a plume configuration comprising a gas constituent distribution in response to the gas monitoring data, and providing a plume communication in response to the plume configuration. Certain further operations of an example procedure are described following, any one or more of which may be performed with the example procedure. An example operation includes determining the plume configuration in response to a personnel description. An example operation includes determining the plume configuration in response to operational information for the facility. An example operation includes determining the plume configuration in response to temperature data. An example operation includes determining the plume configuration in response to weather data. An example operation includes determining the plume configuration in response to the gas constituent distribution for a plurality of gases.

A cohesive control user interface for control of a safety program for hazardous environments is disclosed. Exchange processing time is conserved with automated exchange tracking and processing. Incident reporting time is reduced with insight driven bump, calibration, alarm, and exposure dashboards, as well as custom rule alarm aggregation and alarm heat maps. Time is saved deploying area monitors at a site with interactive map tools/UIs for hazard mapping and device placement. In order to provide users visibility into fleet readiness and worker safety, front-end dashboards are focused on descriptive analytics. These dashboards address gaps in the industrial hygienist incident closeout workflows and equipment managers needs for gas monitor deployment, and are further disclosed herein.

Disclosed herein are tools for interactive device placement, including tools that provide recommendations on where to immediately deploy fixed monitors to address coverage gaps. Recommendations may dynamically change based on weather and hazard changes.

Disclosed herein is an alarm by severity dashboard with alarm noise filtering. The Alarm by Criticality Report Dashboard includes gas monitoring alarms listed and trended by gas type, severity and time.

Disclosed herein is an alarm heat map including a map overlay of current worker location and areas where gas alarms have occurred most frequently.

Disclosed herein is a Bump Test Dashboard. The Bump Test Dashboard provides insights on devices used without bump testing, failed bump tests, equipment group filtering and bump test compliance over time.

Disclosed herein is a Calibration Dashboard including summary statistics on calibration failures by device, calibration compliance, and trending.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems herein. The terms computer, computing device, processor, circuit, and/or server, (“computing device”) as utilized herein, should be understood broadly.

An example computing device includes a computer of any type, capable to access instructions stored in communication thereto such as upon a non-transient computer readable medium, whereupon the computer performs operations of the computing device upon executing the instructions. In certain embodiments, such instructions themselves comprise a computing device. Additionally or alternatively, a computing device may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware and/or computing devices include, without limitation, a general-purpose computer, a server, an embedded computer, a mobile device, a virtual machine, and/or an emulated computing device. A computing device may be a distributed resource included as an aspect of several devices, included as an interoperable set of resources to perform described functions of the computing device, such that the distributed resources function together to perform the operations of the computing device. In certain embodiments, each computing device may be on separate hardware, and/or one or more hardware devices may include aspects of more than one computing device, for example as separately executable instructions stored on the device, and/or as logically partitioned aspects of a set of executable instructions, with some aspects comprising a part of one of a first computing device, and some aspects comprising a part of another of the computing devices.

A computing device may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer-to-peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information (“receiving data”). Operations to receive data include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first receiving operation may be performed, and when communications are restored an updated receiving operation may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g., where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The methods and/or processes described above, and steps thereof, may be realized in hardware, program code, instructions, and/or programs or any combination of hardware and methods, program code, instructions, and/or programs suitable for a particular application. The hardware may include a dedicated computing device or specific computing device, a particular aspect or component of a specific computing device, and/or an arrangement of hardware components and/or logical circuits to perform one or more of the operations of a method and/or system. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above, and combinations thereof, may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer readable instructions described above. All such permutations and combinations are intended to fall within the scope of the present disclosure.

1.-70. (canceled)

71. A system, comprising:
a controller, comprising:
a gas monitor data circuit structured to interpret gas monitoring data from gas monitor end points of a cooperative monitoring group associated with a facility;
a plume characterization circuit structured to determine a plume configuration comprising a gas constituent distribution in response to the gas monitoring data; and
a plume reporting circuit structured to provide a plume communication in response to the plume configuration.

72. The system of claim 71, wherein the plume configuration comprises the gas constituent distribution over a selected spatial region.

73. The system of claim 72, wherein the plume configuration further comprises the gas constituent distribution over a selected temporal region.

74. The system of claim 71, wherein the plume configuration comprises the gas constituent distribution over a selected temporal region.

75. The system of claim 71, wherein the plume configuration comprises a risk overlay over a selected spatial region.

76. The system of claim 75, wherein the plume configuration comprises the risk overlay over a selected temporal region.

77. The system of claim 71, wherein the plume configuration comprises a risk overlay over a selected temporal region.

78. The system of claim 71, wherein the plume characterization circuit is further configured to determine the plume configuration in response to an ambient air flow description.

79. The system of claim 71, wherein the plume characterization circuit is further configured to determine the plume configuration in response to a personnel description.

80. The system of claim 71, wherein the plume characterization circuit is further configured to determine the plume configuration in response to operational information for the facility.

81. The system of claim 71, wherein the plume characterization circuit is further configured to determine the plume configuration in response to temperature data.

82. The system of claim 71, wherein the plume characterization circuit is further configured to determine the plume configuration in response to weather data.

83. The system of claim 71, wherein the plume characterization circuit is further configured to determine the plume configuration in response to the gas constituent distribution for a plurality of gases.

84.-109. (canceled)

110. A method, comprising:
interpreting gas monitoring data from gas monitor end points of a cooperative monitoring group associated with a facility;
determining a plume configuration comprising a gas constituent distribution in response to the gas monitoring data; and
providing a plume communication in response to the plume configuration.

111. The method of claim 110, further comprising determining the plume configuration in response to a personnel description.

112. The method of claim 110, further comprising determining the plume configuration in response to operational information for the facility.

113. The method of claim 110, further comprising determining the plume configuration in response to temperature data.

114. The method of claim 110, further comprising determining the plume configuration in response to weather data.

115. The method of claim 110, further comprising determining the plume configuration in response to the gas constituent distribution for a plurality of gases.
